(19) United States
(12) Plant Patent Application Publication
(10) Pub. No.: US 2002/0002717 P1
(43) Pub. Date: Jan. 3, 2002

(54) APPLE ROOTSTOCK NAMED 'JM1'

(75) Inventors: YOSHIO YOSHIDA, EBETSU-SHI (JP); SHICHIRO TSUCHIYA, RYUGASAKI-SHI (JP); JUNICHI SOEJIMA, MORIOKA-SHI (JP); SHOSUKE SADAMORI, MINAMIAZUMI-GUN (JP); TADAYUKI HANIUDA, NAGANO-SHI (JP); TETSURO SANADA, MORIOKA-SHI (JP); YOSHIKI KASHIMURA, TSUKUBA-SHI (JP); TETSUO MASUDA, MORIOKA-SHI (JP); HIDEO BESSHO, HIGASHIYATSUSHIRO-GUN (JP); SADAO KOMORI, ISHIGAKI-SHI (JP); YUJI ITO, NAKA-GUN (JP)

Correspondence Address: ARMSTRONG,WESTERMAN, HATTORI,, MCLELAND & NAUGHTON, LLP, 1725 K STREET, NW, SUITE 1000, WASHINGTON, DC, 20006, US

(73) Assignee: National Institute of Fruit Tree Science Ministry of Agriculture, Forestry and Fisheries, Tsukuba-shi (JP)

(21) Appl. No.: 09/271,370

(22) Filed: Mar. 18, 1999

(51) Int. Cl. A01H 5/00

(52) U.S. Cl. PLT/174

(57) ABSTRACT

Disclosed herein is an apple tree which can be used as an apple dwarfing rootstock. The apple rootstock is capable of reproduction from hardwood cuttings, exhibits excellent disease and insect resistance, particularly against crown rot, Alternaria blotch, scab and woolly apple aphid, has dwarfing capability and good graft compatibility, exhibits satisfactory growth and grows to a size usable as a rootstock in one season after cuttings, and is therefore excellent as an apple dwarfing rootstock. The apple rootstock tree bears small size fruit, having a oblate shape and reddish yellow colored skin.

### BACKGROUND OF THE VARIETY

The present invention relates to a new and distinct variety of apple rootstock, specifically a dwarfing rootstock variety of apple tree which is capable of reproduction from hardwood cuttings and which exhibits excellent disease and insect resistance, environmental adaptability and graft compatibility.

In Japan, cultivation of dwarfed apple trees to date occupies 23% of the total cultivation area, but many problems remain to be solved. The dwarfing apple rootstocks mainly used in Japan at the present time are ‘M.26’ and ‘M.9’, but these M-line dwarfing rootstocks are associated with such problems as outbreak of violet root rot, cankers and crown rot, and weak tree vigor due to development of burrknots. Also, because the M-line rootstocks cannot be propagated by hardwood cuttings in the manner of the traditional ‘Maruba Kaido’ (Malus prunifolia BORKH. var. ringo Asami), it is common in Japan to graft M-line stocks on to ‘Maruba Kaido’ for use as double stocks. For this reason, extra costs are incurred for nursery stock production, while high growth after planting prevents adequate performance of the characteristics of the dwarfing rootstocks.

It is an object of the present invention to improve these disadvantages of current dwarfing rootstock varieties, and to raise a rootstock variety which is capable of reproduction from hardwood cuttings and which exhibits excellent disease and insect resistance, environmental adaptability, graft compatibility and dwarfing capability.

### Origin and Asexual Reproduction of the Variety

The new variety of apple tree according to the invention was obtained in 1972 in the form of crossed seeds from pollination of ‘Maruba Kaido’ with ‘M.9’, which had been cultivated at the Morioka Branch of the Fruit Tree Research Station (currently the Apple Research Center, the National Institute of Fruit Tree Science), and after seeding the following year and raising at a nursery, the seedlings were selected based on a bark/wood ratio of the roots more than 60% and a propagability by hardwood cuttings more than 50%, and a noteworthy individual was selected in 1984.

The breeders asexually reproduced the individual from hardwood cuttings, as a strain number ‘Apple Rootstock Morioka No. 1’, and from 1985 began the local adaptability test of the rootstock at 12 testing centers in apple producing regions such as Hokkaido, Aomori prefecture, Akita prefecture, Yamagata prefecture and Nagano prefecture.

The excellent results were obtained from the trials, on such characteristics as hardwood cutting reproduction ability as well as dwarfing capability and disease/insect resistance, while its homogeneity and stability were also confirmed, as was its distinctness from the parent variety ‘Maruba Kaido’, ‘M.9’ and the control variety ‘M.26’; in 1996 the ‘Apple Rootstock Morioka No. 1’ was named as the new variety ‘JM1’. The genus and species of this tree is ‘Malus prunifolia var. ringo×Malus pumila var. paradisiaca’. This new apple variety ‘JM1’ was filed on Mar. 29, 1996 under the filing number 8722 according to the Seeds and Seedlings Law of Japan.

The original tree of this apple variety is held at the Apple Research Center of the National Institute of Fruit Tree Science, Ministry of Agriculture, Forestry and Fisheries, residing at 92 Nabeyashiki, Shimokuriyagawa, Morioka City, Japan.

### SUMMARY OF THE VARIETY

The new variety of apple tree has moderate vigor, and exhibits ‘spreading’. The roots have a relatively excess water tolerance. The survival rate was as high as 93%, facilitating reproduction from hardwood cuttings. The current shoot resulting from the hardwood cuttings was upright and exhibited satisfactory growth, reaching a size which was usable for a rootstock in one season. Resistance to major diseases included crown rot, Alternaria blotch and scab. It exhibited medium resistance to fire blight. It also exhibited resistance to the major pest, woolly apple aphid. No susceptibility was exhibited for the top-working disease virus, ASPV (apple stem pitting virus), but it was susceptible to ACLSV (apple chlorotic leaf spot virus).

The ripening period for the fruit is in middle to late September. The fruit averages about 25 g and is quite small. The fruit skin color is deep yellow. It is extremely acidic with a stringent taste, making it unsuitable for eating consumption.

When the variety is used as a rootstock it exhibits dwarfing ability comparable to ‘M.9 EMLA’. Its grafting compatibility with ‘Fuji’ is “good”, and it exhibits slight overgrowth of the rootstock. The production efficiency is higher than ‘M.9 EMLA’ or ‘M.26 EMLA’, giving an abundant yield.

The fruits produced using this variety as the rootstock have a high ratio of large and medium fruits and are 2-3% heavier than those using the control rootstocks ‘M.9 EMLA’ and ‘M.26 EMLA’. High quality fruit is produced, with an equivalent or a higher firmness and sugar content than the control stocks, and color of “fair” to “good”.

It is distinguished from the fruit of the control stock ‘M.26’ by having a longer ripening period, smaller fruit and stronger acidity, being capable of reproduction from hardwood cuttings, and being resistant to woolly apple aphid. It is also distinguished from the fruit of the pollen parent ‘M.9’ by having a longer ripening period, smaller fruit and stronger acidity, being capable of reproduction from hardwood cuttings, and being resistant to wooly apple aphid. Also, it is distinguished from the seed parent ‘Maruba Kaido’ by having a thick and upright current shoot, and by having dwarfing ability when utilized as a rootstock.

The color values presented are herein taken from the Japanese Horticultural Plant Standard Color Chart (JHSC).

### BRIEF DESCRIPTION OF THE DRAWINGS

FIG. 1 is a photograph showing the state of the growth and development of the new variety of apple rootstock after hardwood cuttings;

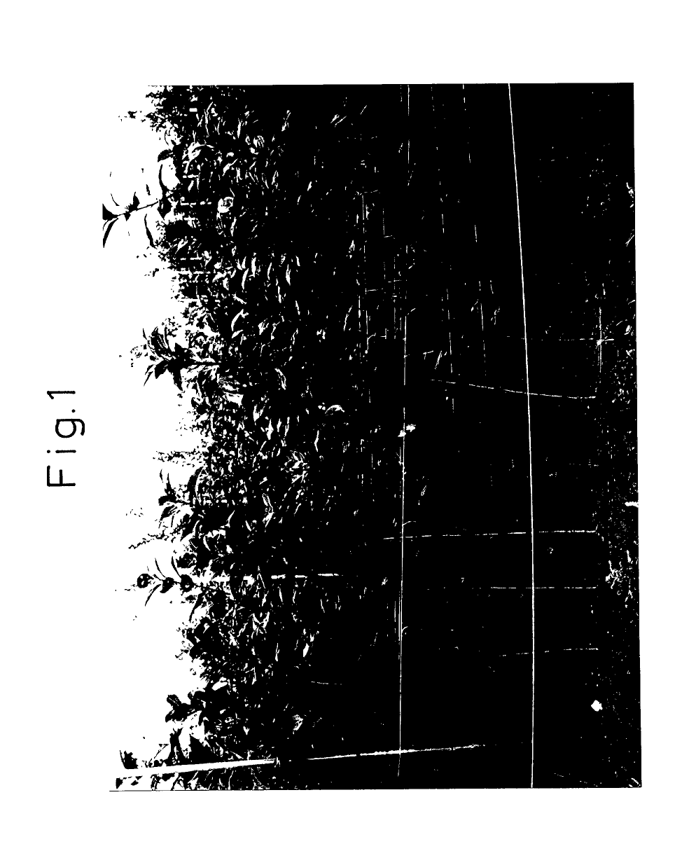

FIG. 2 is a photograph of adult leaves (upper and reverse side) of the new variety of apple rootstock;

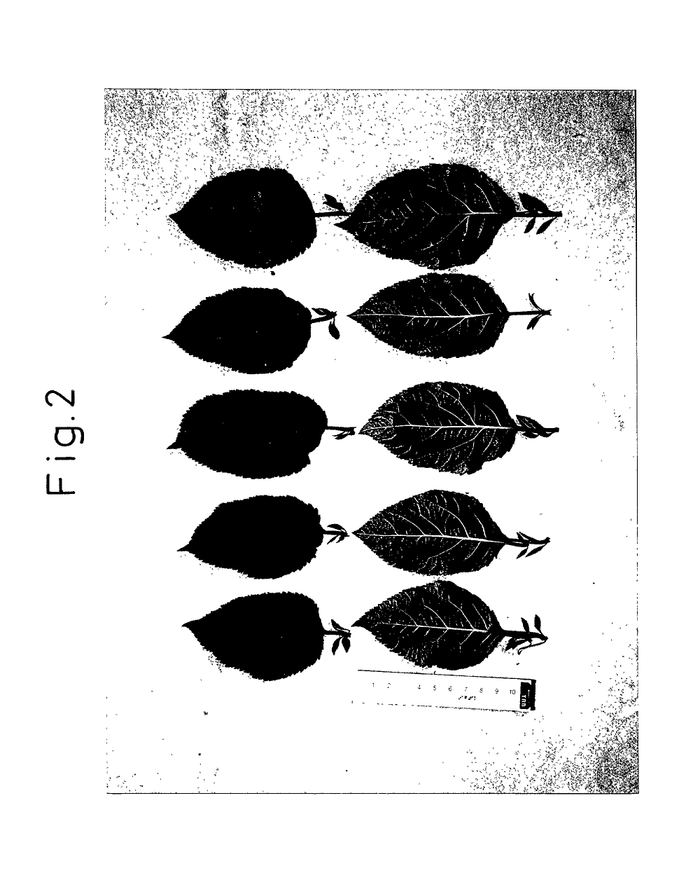

FIG. 3 is a photograph of opened flowers (the top rank and the second rank) and unopened flowers (the third rank and the fourth rank) of the new variety of apple rootstock;

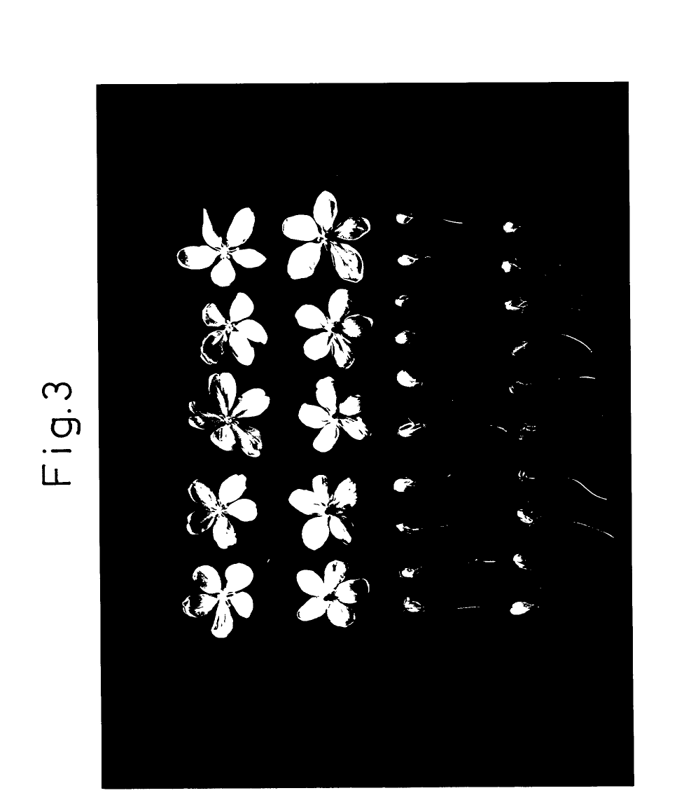

FIG. 4 is a photograph of the fruits on the tree of the new variety of apple rootstock;

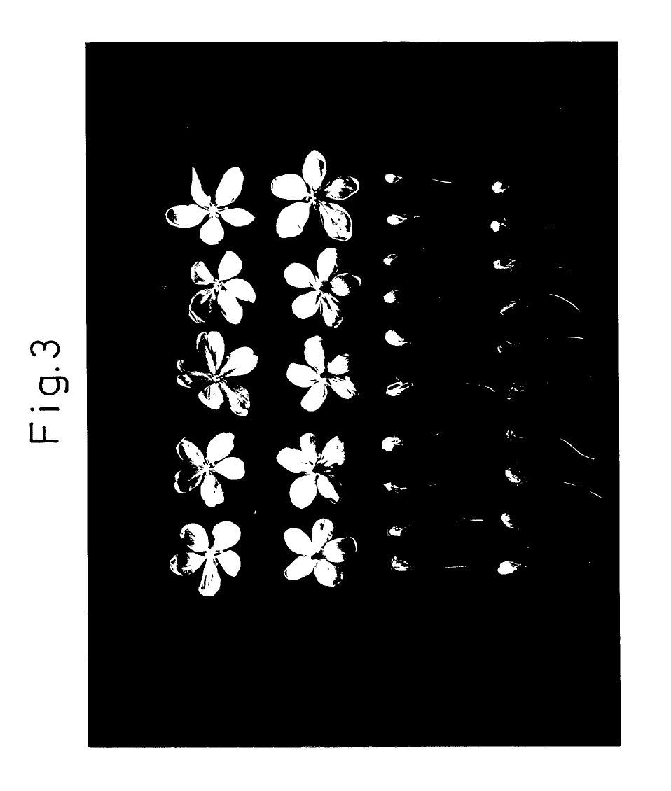

FIG. 5 is a photograph of side views of the fruit of the new variety of apple rootstock;

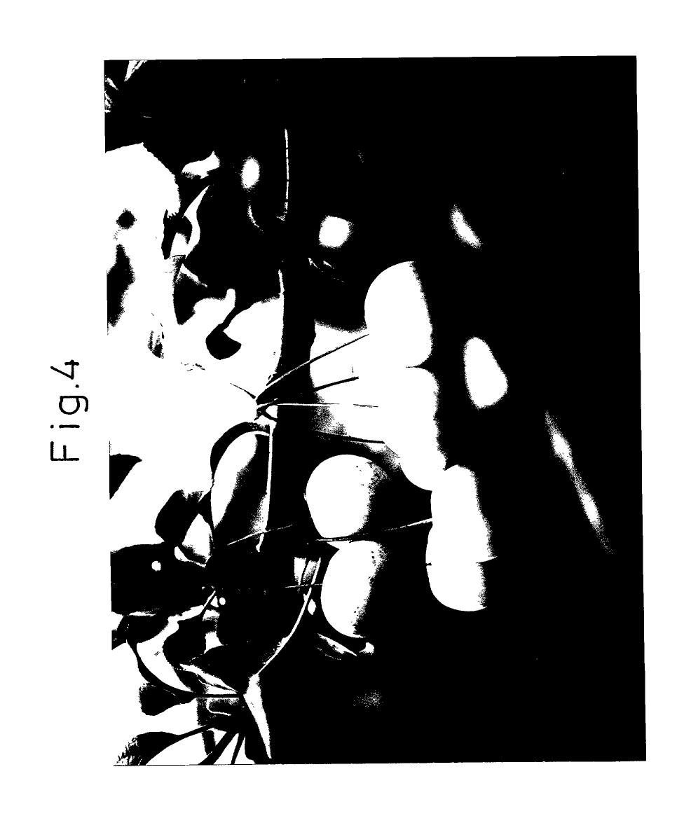

FIG. 6 is a photograph of longitudinal-sectional views of the fruit of the new variety of apple rootstock;

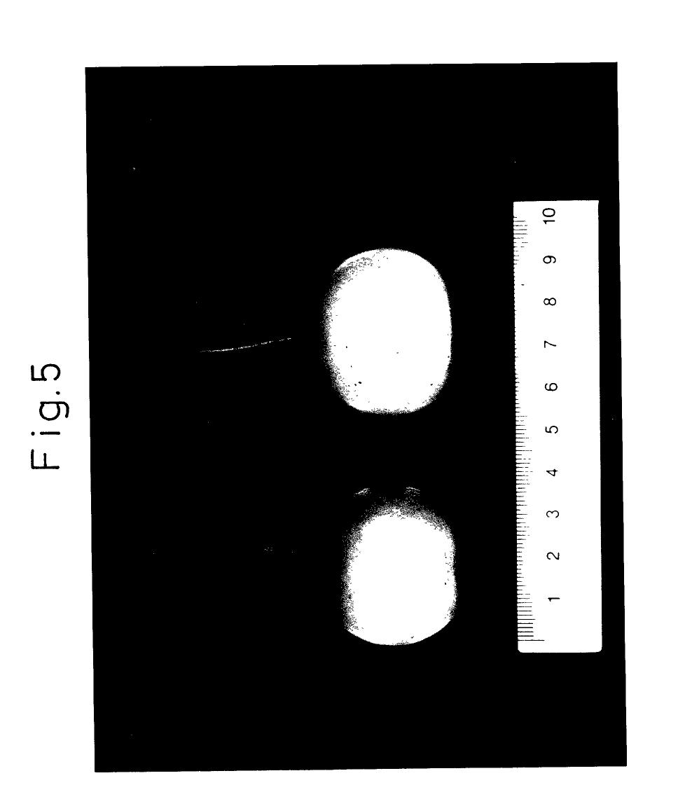

FIG. 7 is a photograph of cross-sectional views of the fruit of the new variety of apple rootstock.

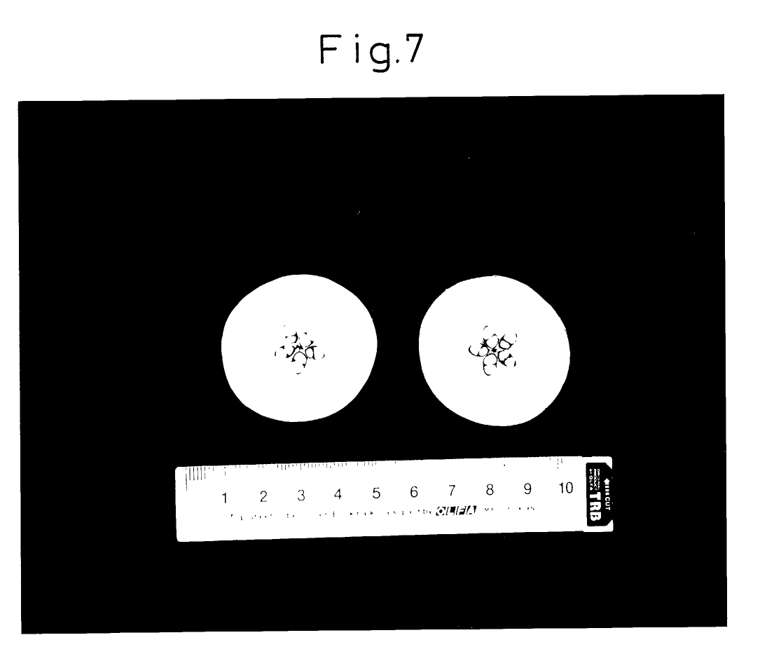

### DESCRIPTION OF THE VARIETY

The characteristics of the new and distinct variety of apple rootstock ‘JM1’ are follows:

Tree:

Habit of branches.—Spreading.

Size.—Small.

Vigor.—Medium.

Thickness (one-year-old shoot).—Stout.

Length of internode (one-year-old shoot).—Medium (about 2.7 cm).

Size of lenticels (one-year-old shoot).—Medium.

Number of lenticels (one-year-old shoot).—Many.

Pubescence of shoot.—Absent or very weak.

Bearing habit.—On spur.

Bearing of axillary flower bud.—Few.

Leaf:

Shape of leaf blade.—Medium.

Serrations of leaf edge.—Serrated.

Size of leaf (length).—Long (length about 10.4 cm, wide about 5.9 cm).

Color of leaf.—Dark green.

Pubescence of leaf.—Few.

Shape of stipules.—Medium.

Length of stipules.—Long.

Length of petiole.—Short.

Thickness of petiole.—Stout.

Flowers:

Number of flowers (per cluster).—Medium.

Size (open flower).—Large.

Color (unopened flower).—Deep pink (JHSC 0106).

Shape of petal.—Oval.

Number of petals.—Medium (5).

Number of stamens.—Medium.

Color of anthers.—Bright yellow.

Amount of pollen.—Much.

Fruit:

Shape.—Oblate, same as ‘M.26’ and ‘M.9’.

Crowning at eye end.—Weak.

Aperture of eye.—Closed.

Depth of basin (eye end).—Shallow.

Breadth of basin (eye end).—Narrow.

Depth of stalk cavity.—Shallow.

Breadth of stalk cavity.—Medium.

Size.—Very small (about 25 g), smaller than ‘M.26’ (about 113 g) and ‘M.9’ (about 43 g).

Type of ground color.—Reddish yellow (JHSC2210).

Type of over color of skin.—Reddish yellow.

Intensity of over color.—Deep, whereas that of ‘M.9’ is pale.

Form of over color of skin.—Solid (undistinguished stripe).

Amount of over color of skin.—Few.

Position of russet.—

Amount of russet.—Absent or very slight.

Raised russet lenticels.—Absent, same as in ‘M.9’.

Size of lenticels.—Small.

Number of lenticels.—Many.

Silvery mottle of skin (scarfskin).—Absent.

Shininess of skin.—Strong.

Greasiness of skin.—Medium.

Bloom of skin.—Absent.

Cracking tendency of skin.—Present.

Surface texture of skin.—Smooth.

Length of stalk.—Long (about 5.0 cm), longer than ‘M.26’ and ‘M.9’.

Thickness of stalk.—Slender, slightly more slender than ‘M.9’ and ‘M.26’.

Distinct swelling at end of stalk.—Absent.

Shape of core.—Flat round.

Size of core.—Medium (5 core cells).

Color of flesh.—Yellowish white, that of ‘M.9’ is white.

Easy bruising of flesh.—Medium.

Browning of flesh.—Strong.

Firmness of flesh.—Medium.

Texture of flesh.—Coarse.

Water core.—Absent or weak.

Sweetness of flesh.—Medium (about Brix 14.0°), stronger than ‘M.9’ (about Brix 8.4°).

Acidity of flesh.—Strong (about 1.18%), stronger than ‘M.9’ and ‘M.26’.

Astringency of flesh.—Present.

Flavor of flesh.—Weak.

Juiciness of flesh.—Medium.

Seed:

Number of fully developed seeds.—Medium (average 6.3).

Shape.—Ovovoid.

Size.—Small.

Physiological and ecological characteristics:

Date of germination.—Early (April 7th at Morioka City, Japan).

Season of flowering.—Medium (Around May 13th, at Morioka City, Japan).

Season of leaf fall.—Medium.

Time fruit are ripe for eating.—Early (Around September 17 to 23th, at Morioka City, Japan), same as ‘M.9’ (Around September 17th) and ‘M.26’ (September 24th).

Predominancy of precocity.—Early.

Self-fruitfulness.—Medium (about 29%).

Early dropping of fruit.—Medium.

Preharvest dropping of fruit.—Medium.

Physiological disorder of fruit.—Few.

Keeping quality of fruit (room condition).—Short (about 7 days).

Keeping quality of fruit (cold storage).—Short (about 30 days).

Occurrence of heart rot (core rot).—Absent or weak.

Resistance to Alternaria blotch.—Resistant as same as ‘M.9’.

Resistance to powdery mildew.—Susceptible.

Resistance to rough bark disorder.—Resistant.

Resistance to aphid.—Susceptible.

Resistance to woolly apple aphid.—Resistant, whereas ‘M.26’ and ‘M.9’ are susceptible.

Grafting.—Easy.

Rooted cuttings.—Easy, whereas hard in ‘M.26’ and ‘M.9’.

Vegetative propagation.—Easy, easier than ‘M.26’ and ‘M.9’.

Habit of tree vigor after grafting.—Moderate, and shows a dwarfing ability as a rootstock.

Development of roots.—Medium.

Excess water tolerance.—Strong, stronger than ‘M.26’ and ‘M.9’.

Drought resistance.—Medium.

Culture: Since the new variety is susceptible to the top-working disease virus ACLSV, it succumbs to the disease when grafted with a carrier scion. Therefore, it is necessary to use scions free from ACLSV.

For planting of nursery stock, support posts are required to prevent lodging, as for dwarfed trees of ‘M.9’ and ‘M.26’.

Because the new variety has superior qualities as an apple dwarfing rootstock, it is suitable for use in all cultivation zones for apples, and it is believed that it will rapidly come into wide use in place of the currently used rootstocks, ‘M.9’ and ‘M.26’.

1. A new and distinct variety of apple rootstock, substantially as described in the present specification, characterized by being capable of reproduction from hardwood cuttings, exhibiting excellent resistance to crown rot, Alternaria blotch, scab and woolly apple aphid, having dwarfing capability and good graft compatibility, exhibiting satisfactory growth, reaching a size which is usable as a rootstock in one season after cuttings and being therefore useful as an apple dwarfing rootstock, and bearing a small size fruit, having a oblate shape and reddish yellow colored skin.
